(19) United States
(12) Patent Application Publication
(10) Pub. No.: US 2008/0119860 A1
(43) Pub. Date: May 22, 2008

(54) System for preparing bone for receiving an implant

(75) Inventor: Thomas Francis McCarthy, Neshanic Station, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/603,317

(22) Filed: Nov. 21, 2006

(51) Int. Cl. A61F 5/00; A61B 17/00; A61B 17/32

(52) U.S. Cl. 606/87; 606/82; 606/169

(57) ABSTRACT

A system for cutting bone includes a cutting guide including a metal block having a guide surface provided thereon, and an oscillating tip saw having a cutting blade assembly extending from a distal end thereof. At least one element of the cutting blade assembly is magnetic for generating a magnetic attraction to the guide surface of the cutting guide so as to control the orientation of the cutting blade assembly during a bone cutting procedure. The cutting guide may also be magnetic.

### FIELD OF THE INVENTION

The present invention generally relates to surgical tools and procedures and more specifically relates to surgical saws and cutting guides used for preparing bone sites for receiving implants.

### BACKGROUND OF THE INVENTION

Prosthetic implants are frequently used to replace damaged natural joints in the body. During a surgical procedure, in order to prepare a stable implant site, it is usually necessary to remove additional bone beyond the damaged portion of the joint.

During orthopedic implant procedures, saws are often used to selectively remove bone. During such procedures, it is very important to form precise cut lines in bone because the prosthetic implant parts are designed to fit snuggly in the prepared bone site. In order to insure that precise cut lines are formed, surgeons typically mount a cutting guide or cutting block to the bone adjacent to the location where the cut is to be made. One type of cutting guide has a precisely shaped set of slots that define the cut lines through the bone. After the cutting guide has been secured to bone, the surgeon prepares the bone site by sequentially inserting the saw blade in the slots and cutting the bone along the precisely defined lines.

One type of saw used during orthopedic surgical procedures is a sagittal saw. A sagittal saw generally includes a handpiece that houses a motor and a control circuit that regulates actuation of the motor. Extending distally from the handpiece is a planar saw blade, and the most distal end of the saw blade has teeth for cutting hard tissue such as bone. The sagittal saw includes a drive mechanism inside the housing that transfers the power developed by the motor to the saw blade. The drive mechanism converts the rotary motion produced by the output shaft of the motor to the blade so that the blade oscillates back and forth in the plane of the saw blade for cutting bone.

Conventional saws and cutting guides work reasonably well, however, there are some noticeable limitations. For example, as the cutting blade oscillates during a cutting procedure, the blade rubs against the material bounding the slots of the cutting guide. This repetitive contact wears away the slot-defining material, which widens the slot so much that it can no longer serve to precisely define cut lines through bone. Another disadvantage of the cutting blade engaging the side walls of the slot is that the extra power required to overcome friction forces will reduce the life of the battery powering the saw. Thus, the surgical procedure may be lengthened if the battery has to be changed.

In addition, the wearing of the material defining the slots of the cutting guide generates a fine dust that settles at the surgical site. As a result, surgical personnel are required to flush the site to remove the dust. Having to repeatedly perform this flushing process will also lengthen the surgical procedure and the amount of time the patient is subject to anesthesia. Furthermore, the oscillating motion of the blade causes vibration of the saw. As a result, the surgeon is required to engage in some conscious or unconscious physical effort to hold the saw steady when it vibrates. Over time, having to hold the saw steady to overcome this vibration can be mentally and physically draining.

One objective of implant surgery is to perform the procedure using a minimally invasive surgical (MIS) practice. When performing a minimally invasive procedure, only a relatively small opening is made in the soft tissue surrounding the bone or the joint. As a result, the oscillating saw blade used in a bone resection procedure is typically longer than the saw blade used to perform a conventional resection. The longer blade has a mass moment of inertia that is greater than the mass moment of inertia associated with shorter-length blades. Consequently, when the saw is actuated, more vibration motion is created by the longer blade than when a shorter blade is used. This increased vibration can make it difficult for a surgeon to hold the saw steady. Moreover, a longer blade is more flexible than a shorter blade, which can result in the longer blade making less precise cuts in the bone.

In spite of the above advances, there remains a need for improved saws and cutting guides for preparing implant sites in bone. There also remains a need for improved tools and methods for making precise cuts through bone.

### SUMMARY OF THE INVENTION

In certain preferred embodiments of the present invention, a system for cutting bone includes a cutting instrument and a cutting guide for guiding the cutting instrument as the cutting instrument advances through bone. The cutting instrument may be any type of cutting instrument that is used in surgical procedures for cutting bone. In certain preferred embodiments, the cutting instrument may be a saw including a saw blade having cutting teeth disposed along an edge of the saw blade. The saw blade is preferably magnetic for being magnetically attracted to a guide surface on the cutting guide, as will be described in more detail below. In other preferred embodiments, the cutting instrument may be a power tool such as a reciprocating saw or an oscillating tip saw. In these embodiments, at least one element of the saw is magnetic for being magnetically attracted to the guide surface of the cutting guide.

The cutting guide may be made of metal and may include openings that receive fasteners for anchoring the cutting guide to bone. The cutting guide includes at least one guide surface for guiding advancement of the cutting instrument into bone during a surgical procedure. The guide surface is preferably planar. The cutting guide is preferably made of metal for attracting the magnetic component on the cutting instrument to the guide surface on the cutting guide. In certain preferred embodiments, both the cutting instrument and the cutting guide may be magnetic for generating the magnetic attraction between the two components.

In other preferred embodiments, the cutting instrument is an oscillating tip saw having a cutting blade assembly and an oscillating cutting tip. The cutting blade assembly preferably has one or more magnetic elements that are magnetically attracted to the cutting guide. In certain preferred embodiments, at least one magnetic element of the cutting blade assembly is a guide bar assembly that supports the oscillating cutting blade. In certain embodiments, the oscillating cutting tip is non-magnetic. In other embodiments, however, the oscillating tip may be magnetic so that the location of the tip can be tracked during surgery.

In certain preferred embodiments, the cutting instrument includes a guide bar assembly having a proximal end and a distal end, and a cutting blade pivotally mounted to the guide bar assembly, whereby the cutting blade projects beyond the distal end of the guide bar assembly. The cutting instrument preferably includes at least one drive element coupled with the cutting blade for selectively oscillating the cutting blade. The cutting blade desirably has cutting teeth for cutting into tissue or bone. In one embodiment, the guide bar assembly is magnetic and the cutting blade is non-magnetic. In this embodiment, the at least one drive element is non-magnetic. In another embodiment, the guide bar assembly and the cutting blade are magnetic and the at least one drive element is non-magnetic.

In certain preferred embodiments, the guide bar assembly desirably includes a magnetic bottom bar, a magnetic top bar spaced from the bottom bar, and a non-magnetic inner bar disposed between the bottom bar and the top bar, whereby the cutting blade projects from distal ends of the bottom bar and the top bar. The at least one drive element desirably includes a pair of non-magnetic drive rods that extend between the bottom bar and the top bar along opposite lateral sides of the inner bar.

In certain preferred embodiments, the magnetic elements are made of metals that may be magnetized such as steel, iron, nickel, cobolt and alloys containing any one of the above-listed elements. As is well known to those skilled in the arts, the metals may be magnetized by exposing the metals to magnetic fields or by placing the metals next to magnets. In other preferred embodiments, the metals may be magnetized by wrapping a wire around a piece of metal that may be magnetized and passing an electrical current through the wire to form an electromagnet.

In certain preferred embodiments of the present invention, the cutting instrument includes a housing, a motor located in the housing and coupled with the cutting blade, and an actuator in communication with the motor for selectively activating the motor for driving oscillating movement of the cutting blade. The cutting instrument also desirably includes a drive shaft connected with the motor and coupled with the cutting blade, whereby the drive shaft drives oscillating movement of the cutting blade when the motor is activated.

In another preferred embodiment of the present invention, a system for cutting bone includes a cutting guide including a metal block having a guide surface provided thereon, and an oscillating tip saw having a cutting blade assembly extending from a distal end thereof. At least one element of the cutting blade assembly is magnetic for generating a magnetic attraction to the guide surface so as to control the orientation of the cutting blade assembly during a bone cutting procedure. The cutting blade assembly desirably includes a magnetic guide bar assembly and a non-magnetic cutting blade pivotally connected with the magnetic guide bar assembly. The cutting blade assembly also desirably includes at least one non-magnetic drive element extending through the guide bar assembly for driving oscillating movement of the cutting blade.

In still another preferred embodiment of the present invention, a system for cutting bone includes a plurality of cutting guides having different sizes, each cutting guide including a metal block having a guide surface provided thereon. The system preferably includes an oscillating tip saw having a cutting blade assembly extending from a distal end thereof, the cutting blade assembly having an oscillating cutting blade provided at a distal end thereof, whereby at least one element of the cutting blade assembly is magnetic for generating a magnetic attraction between at least one magnetic element and the guide surfaces of the cutting guides. In certain preferred embodiments, the metal blocks are magnetic. The oscillating tip saw preferably includes a housing, a motor disposed in the housing and coupled with the cutting blade assembly for selectively driving the oscillating cutting blade, and an actuator provided on the housing and coupled with the motor for activating the oscillating cutting blade.

In yet a further preferred embodiment of the present invention, a system for cutting bone includes a cutting guide having a guide surface, a cutting instrument including an oscillating portion and a non-oscillating portion. The oscillating portion of the cutting assembly is non-magnetic and the non-oscillating portion of the cutting assembly is magnetic for attracting the non-oscillating portion of the cutting assembly to the guide surface of the cutting guide for guiding the cutting assembly over the guide surface when cutting bone. In certain preferred embodiments, the cutting instrument includes an oscillating tip saw. The oscillating portion of the saw desirably includes a cutting blade having cutting teeth. In certain embodiments, the oscillating portion of the cutting instrument is non-magnetic.

These and other preferred embodiments of the present invention will be described in more detail below.

### BRIEF DESCRIPTION OF THE DRAWINGS

FIG. 1 shows a perspective view of an oscillating tip saw, in accordance with certain preferred embodiments of the present invention.

FIG. 2 shows an exploded view of the oscillating tip saw shown in FIG. 1.

FIG. 3 shows a side cross-sectional view of the oscillating tip saw shown in FIG. 1.

FIG. 4 shows a top cross-sectional view of the oscillating tip saw shown in FIG. 3.

FIG. 5 shows a perspective view of a drive base of the oscillating tip saw shown in FIG. 2.

FIG. 6 shows a top plan view of a drive link of the oscillating tip saw shown in FIG. 2.

FIG. 7A shows a plan view of a saw blade assembly of the oscillating tip saw shown in FIG. 1.

FIG. 7B shows a side cross-sectional view of a portion of the saw blade assembly shown in FIG. 7A.

FIG. 8 shows an exploded view of the saw blade assembly of the oscillating tip saw shown in FIG. 1.

FIG. 9 shows a plan view of a cutting blade of the saw blade assembly shown in FIG. 8.

FIG. 10 shows a perspective view of the oscillating tip saw of FIG. 1 and a cutting guide, in accordance with certain preferred embodiments of the present invention.

FIG. 11 shows a perspective view of a cutting guide for guiding a saw during a surgical procedure, in accordance with another preferred embodiment of the present invention.

FIGS. 12A-12B show a method of preparing a bone site using a magnetized saw blade in conjunction with the cutting guide shown in FIG. 11.

FIG. 13 shows a kit including a plurality of cutting guides having different sizes, in accordance with certain preferred embodiments of the present invention.

FIG. 14 shows a magnetic cutting guide, in accordance with certain preferred embodiments of the present invention.

### DETAILED DESCRIPTION

In the present application, certain terminology is used for describing the invention and should not be construed as limiting the scope of the invention. For example, the words “upwardly”, “downwardly”, “rightwardly” and “leftwardly” will refer to directions in the drawings to which reference is made. The words “inwardly” and “outwardly” refer to directions toward and away from, respectively, the geometric center of what is shown in the drawings. The word “distally” means the end of a tool that is closer to a patient, and the word “proximally” means the end of the tool that is further away from the patient. The above terminology includes the words specifically mentioned, derivatives thereof, and words of similar import.

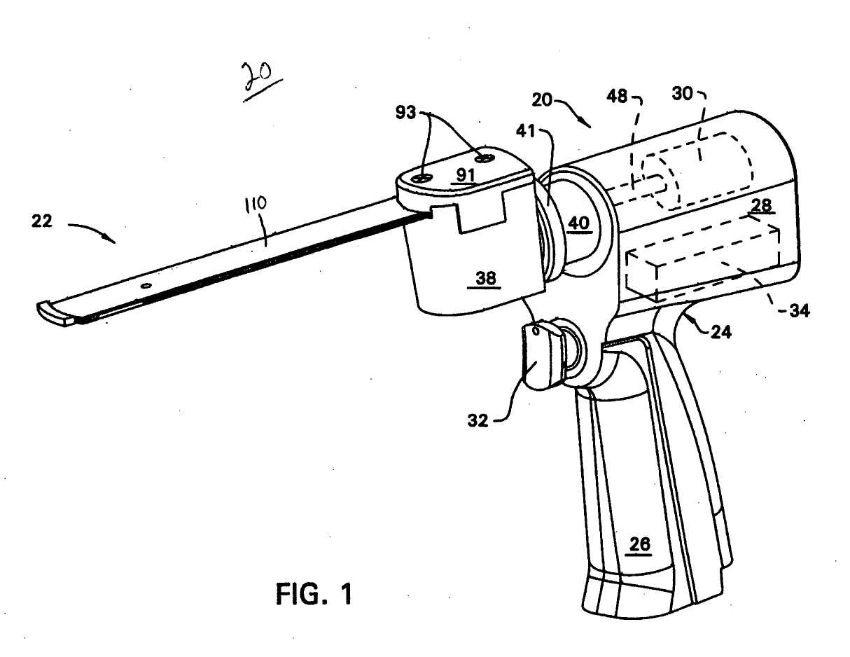

Referring to FIG. 1, in accordance with certain preferred embodiments of the present invention, an oscillating tip saw 20 includes a cutting blade assembly 22 and a housing 24 that is attached to the cutting blade assembly. As will be described in more detail below, at least one element of the cutting blade assembly is magnetic for being magnetically attracted to a guide surface on a cutting guide. The magnetic attraction of the cutting blade assembly with the cutting block will prevent the cutting blade assembly from moving off course as it advances through bone.

The housing 24 includes a handgrip 26, an upper shell 28 that extends over the handgrip 26 and a motor 30 provided inside the upper shell. The housing includes a battery compartment (not shown) provided at a lower end of the handgrip 26 that is adapted to receive a battery for powering the oscillating tip saw. The housing 24 also preferably includes a manually retractable trigger 32 that extends distally from the distal surface of the handgrip 26. The oscillating tip saw 20 also preferably includes a control module 34 that is located in the upper shell 28 immediately above the trigger 32 and below the motor 30. The control module 34 has internal electronics, such as microprocessors and/or semiconductor elements, that monitor the extent to which the trigger is depressed and, based upon the position of the trigger, regulate the actuation of the motor 30.

The oscillating tip saw 20 also includes a coupling element 38 that couples the cutting blade assembly 22 with the housing 24. The coupling element 38 is attached to the housing 24 by a cylindrical neck 40 that is attached to the coupling element 38. In certain preferred embodiments, the cylindrical neck 40 is integrally connected with the coupling element 38. The cylindrical neck 40 has an outer surface that includes threads (not shown) so that the neck 40 may be threadably secured to a threaded opening (not shown) formed in the upper shell 28 of the housing.

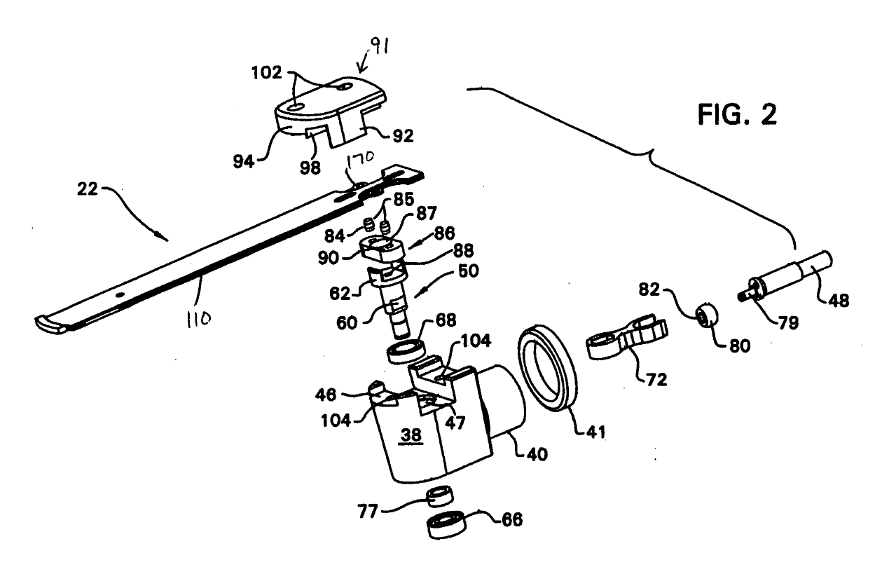

Referring to FIGS. 2-4, the oscillating tip saw 20 includes a locking ring 41 located distally of the upper shell 28. When the components of the housing 24 are assembled together, the coupling element 38 and the cylindrical neck 40 are rotated so as to be in the proper orientation relative to the upper shell 28 of the housing. The locking ring 41 is then rotated around the neck 40 so as to press against the front face of the upper shell 28 for holding the coupling element 38 and the neck 40 in position.

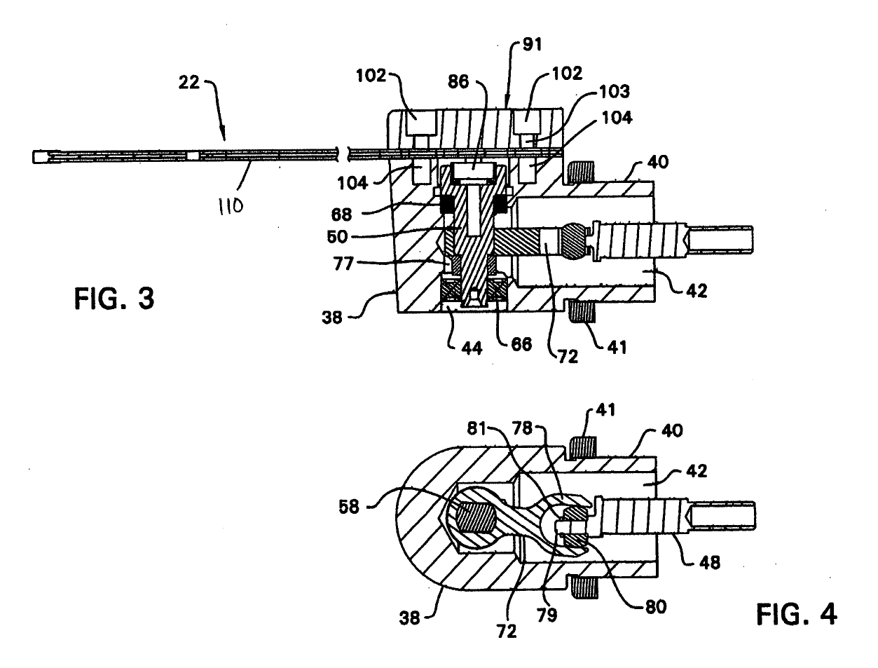

Referring to FIGS. 3 and 4, the coupling element 38 is located at the distal end of the neck 40 and has a cross-sectional width greater than the diameter of the neck 40. The distal face of the coupling element 38 is curved. A first bore 42 extends longitudinally through the cylindrical neck 40 and is in communication with a second bore 44 that extends longitudinally through the coupling element 38. The longitudinal axes of the coupling element 38 and the neck 40 are perpendicularly offset from each other. As a result, the axes of the first and second bores 42 and 44 are perpendicularly offset.

Referring to FIG. 2, the top of the coupling element 38 has two grooves 46 and 47 formed therein. Each of the grooves has a rectangular-shaped cross-section. The first groove 46 extends longitudinally along the coupling element 38, from the distal face toward the neck 40. The second groove 47 extends perpendicular to and crosses the first groove 46. The second groove 47 is deeper than and bisects the first groove 46. Referring to FIG. 3, the second bore 44 in the coupling element 38 extends to the second groove 47.

Referring to FIGS. 2-4, a drive assembly is provided inside the coupling element 38 and the neck 40 for selectively actuating the saw blade assembly 22. The drive assembly includes a drive shaft 48 that extends from the upper shell 28 of the housing and into the neck 40. In certain preferred embodiments of the present invention, the drive shaft 48 may be the actual output drive shaft of the motor 30. In other preferred embodiments, the drive shaft 48 may be an output drive shaft of a speed reduction gear assembly or an idler shaft to which the actual motor drive shaft is connected.

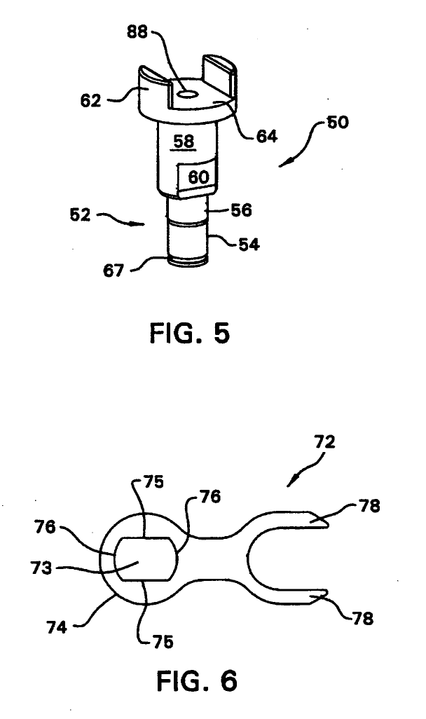

The rotation of the drive shaft 48 oscillates, via a drive link 72, a generally cylindrical, multi-section drive base 50 that is rotatably mounted in the second bore 44 of the coupling element 38. Referring to FIG. 5, the drive base 50 includes a cylindrical stem 52 having a lower section 54 with a first diameter and an upper section 56 with a second diameter that is larger than the first diameter. The drive base 50 also has a main section 58 located above the upper section 56 of the stem having a diameter that is greater than the diameter of the upper section 56. The main section 58 is generally cylindrical and has two diametrically opposed flats 60 at the lower end thereof. The drive base 50 also has a head 62 that is generally circular in shape having an outer diameter greater than the outer diameter of the underlying main section 58. The head 62 of the drive base 50 has a slot 64 that extends in a direction perpendicular to the longitudinal axis of the stem. When the oscillating tip saw is assembled together, the drive base 50 is disposed in the second bore 44 of the coupling element 38 so that the head 62 of the drive base extends into the bottom portion of the second groove 47.

Referring to FIGS. 2 and 3, the oscillating tip saw also includes first and second bearing assemblies 66 and 68 that hold the drive base 50 in the coupling element 38 and enable the drive base to rotate relative to the coupling element. The first bearing assembly 66 extends between the lower section 54 of the stem of the drive base 50 and the adjacent inner wall of the coupling element 38 that defines the second bore 44. The inner race of the first bearing assembly 66 abuts the step between the lower and upper sections 54 and 56 of the stem 52. A retaining ring (not shown) is snap fit into a groove 67 formed in the end of the lower section 54 of the stem for holding the bearing assembly 66 to the stem 52.

The second bearing assembly 68 is positioned around the main section 58 of the stem that is located immediately below the head 62. The second bearing assembly 68 extends between the main section 58 and the inner wall of the coupling element that defines the second bore 44. In certain preferred embodiments, the coupling element is shaped so that the second bore 44 has a large diameter counterbore (not shown). The outer race of bearing assembly 68 seats against the stepped surface between the counterbore and the main sections of the bore 44.

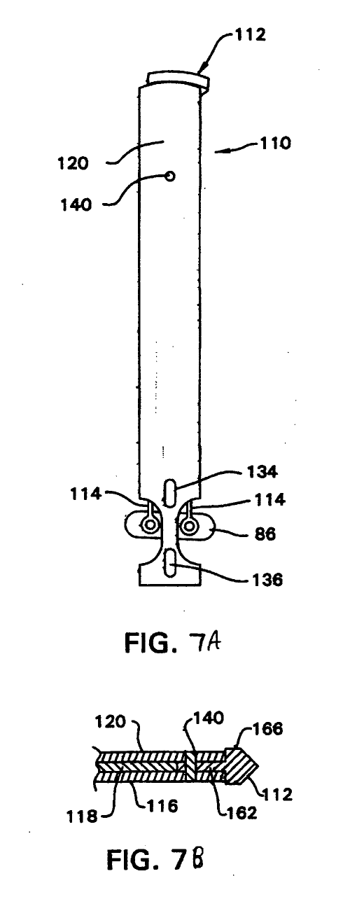

Referring to FIG. 6, the oscillating tip saw also preferably includes a drive link 72 having a circular distal end 74 forming a closed opening 73. The closed opening 73 is defined by two opposed flat walls 75 and two opposed curved walls 76. More particularly, the drive link 72 is shaped so that, when the drive link is fitted to the drive base 50, the flat walls 75 and curved walls 76 that define the opening 73 closely abut the respective flats 60 and adjacent curved surfaces of the main section 58 of the drive base 50. The drive link 72 also has two parallel opposed tines 78 that extend toward the drive shaft 48.

Referring to FIGS. 2 and 3, a collar 77 is press-fit to the upper section 56 of the stem. The collar 77 prevents downward slippage of the drive link 72 from the main section 58 of the drive base 50.

Referring to FIGS. 2-4, the drive shaft 48 has a cylindrical-shaped cam 79 that extends from a distal end of the drive shaft. The cam 79 is parallel to and axially offset from the longitudinal axis of the drive shaft 48. The drive link 72 is positioned relative to the drive shaft so that the cam 79 is disposed between the tines 78 of the drive link 72.

Referring to FIGS. 2 and 4, a spherical bearing 80 has a central opening 82 and the drive shaft cam 79 is insertable into the opening 82. A snap ring 81 holds the bearing 80 to the drive shaft cam 79. The bearing 80 is positioned and dimensioned to slip fit between the drive link tines 78. In operation, the bearing 80 transforms the rotational movement of the cam 79 around the central axis of the drive shaft 48 to an oscillating movement of the drive link 72, and the drive link 72 transfers this oscillating movement to the drive base 50.

Referring to FIGS. 2 and 3, an oscillating bar 86 is secured in the slot 64 formed in the head 62 of the drive base 50. More specifically, a threaded fastener (not shown) extends through an opening 87 formed in the oscillating bar 86 and a bore 88 formed in the drive base 50 for holding the oscillating bar 86 to the drive base 50. The oscillating bar 86 is positioned in the second groove 47 of the coupling element 38. In preferred embodiments, during oscillating movement, the oscillating bar 86 does not contact the opposed interior walls of the second groove 47.

Referring to FIG. 2, two pins 85 are press-fit into separate openings 90 formed in the top surface of the oscillating bar 86. The openings 90 are preferably centered on the longitudinal axis of the oscillating bar 86 and symmetrically located around the opening 87 in the oscillating bar. Each pin 85 preferably has a relatively wide center waist section 84 that limits the extent to which the pins 85 may be press-fit into the separate openings 90 in the oscillating bar 86.

Referring to FIGS. 1-3, the oscillating tip saw includes a cover 91 securable over the top of the coupling element 38 so as to cover the proximal end of the saw blade assembly 22 for coupling the saw blade assembly with the coupling element 38 and the drive assembly described above. The cover 91 preferably includes opposing legs 92 that extend downwardly from opposite sides thereof. The opposing legs 92 are adapted to be seated in opposite ends of the second groove 47 of the coupling element 38.

The cover 91 also includes a downwardly extending center rib 94 that extends along the front of the cover and that is spaced inwardly from the legs 92. When the blade assembly 22 is assembled with the coupling element 38, the proximal end of the blade assembly 22 is seated in the second groove 46 of the coupling element, with the rib 94 being disposed in the second groove 46 above the proximal end of the cutting blade assembly 22. The rib 94 has two parallel opposed flanges 98 that are located on opposite sides of the rib 94 and extend along the length of the rib. When the saw 20 and blade assembly 22 are assembled together, each flange 98 is located between a side edge surface of the blade assembly 22 and the adjacent inner wall of the coupling element 38 that defines the first groove 46.

Referring to FIGS. 1-3, the assembly preferably includes threaded fasteners 93 used for removably securing the cover 91 to the coupling element 38. Each fastener 93 extends through an opening 102 formed with a counterbore 103 (FIG. 3) that extends through the cover 91 and rib 94 into a complementary threaded bore 104 formed in the coupling element 38. The bores 104 extend downwardly from the base of the first groove 46 in the coupling element 38.

Referring to FIGS. 1, 7A-7B and 8, the cutting blade assembly 22 preferably includes a guide bar assembly 110 that extends distally from the coupling element 38. A cutting blade 112 is pivotally connected to the guide bar assembly 110 and extends from the distal end of the guide bar assembly 110. The cutting blade assembly 22 also preferably includes drive elements 114, such as thin, plate-like drive rods, that extend between the oscillating bar 86 (FIG. 2) located in the coupling element 38 and the cutting blade 112. The drive elements 114 transfer oscillating motion from the oscillating bar 86 to the cutting blade 112 so that when the oscillating tip saw 20 is actuated, the cutting blade 112 moves back and forth in an oscillating motion.

In one preferred embodiment, the guide bar assembly 110 is magnetic and the cutting blade 112 is non-magnetic. In these embodiments, the magnetic guide bar assembly 112 is attracted to a guide surface on a metallic cutting guide for properly aligning the guide bar assembly and the cutting blade with bone to be cut. The magnetic attraction preferably minimizes the effects of vibration and maintains the cutting blade along preferred cut lines as the saw advances through the bone. In other preferred embodiments of the present invention, the guide bar assembly 110 and the cutting blade 112 are magnetic and the drive elements 114 are non-magnetic for providing the same benefits of the magnetic attraction discussed above.

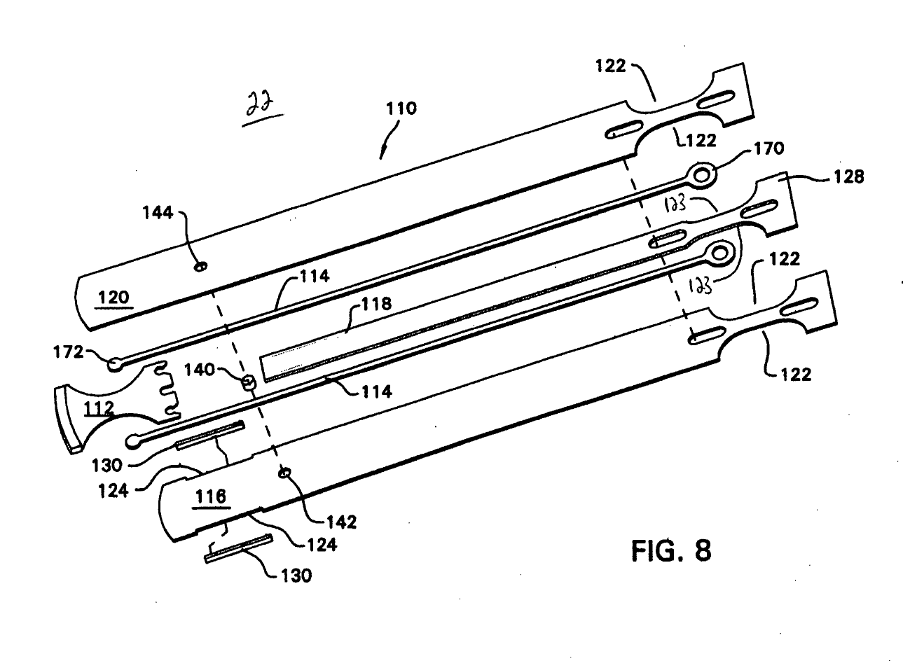

Referring to FIG. 8, the guide bar assembly 110 includes three planar, plate-shaped bars that are assembled together including a bottom bar 116, an inner bar 118 and a top bar 120. The bottom bar 116 and the top bar 120 preferably have the same overall length and width. The proximal ends of the bottom bar and the top bar 116 and 120 have inwardly directed cutouts 122. The distal ends of the bottom bar 116 and the top bar 120 are preferably curved. The bottom bar 116 also has opposed rectangular notches 124 located adjacent the distal end of the bottom bar.

The inner bar 118 is preferably shorter and narrower than the bottom bar 116 and the top bar 120. The proximal end of the inner bar 118 has a stem 128 with an edge surface that is preferably alignable the edge surfaces of the bottom bar 116 and the top bar 120. The proximal end of the inner bar has inwardly directed cutouts 123 that are preferably aligned with the inwardly directed cutouts 122 formed in the bottom bar 116 and the top 120. The inner bar 118 has a constant width between the inwardly directed cutouts 123 and the distal end of the inner bar.

In certain preferred embodiments, the inner bar 118 has a length that is about 70 to 90% of the overall length of the respective bottom and top bars 116 and 120. In more preferred embodiments, the length of the inner bar 118 is between 75 and 85% of the overall length of the respective bottom and top bars 116 and 120. In preferred embodiments, the width of the distal end of the inner bar 118 is between 30 and 95% of the width of the respective bottom and top inner bars 116 and 120. In more preferred embodiments, the width of the distal end of the inner bar 118 is between 50 and 90% of the width of the respective bottom and top bars 116 and 120.

Referring to FIG. 8, the guide bar assembly 110 is assembled by welding a pair of support bars 130 in the notches 124 of the bottom bar 116. The support bars 130 are generally rectangular in shape. One of the support bars 130 is welded in each notch so as to extend upwardly from the bottom bar 116.

During assembly, the inner bar 118 and the top bar 120 are stacked over the bottom bar 116 and the support bars 130 are welded to the abutting inwardly directed face of the top bar 120. Once the guide bar assembly 110 is partially assembled (the inner bar 118 is still loose at this point), the sandwiched metal forming the bottom, inner and top bars 116, 118 and 120 is selectively removed in a single operation to form a generally rectangular guide slot 132 and two oval shaped openings 134 and 136 (FIG. 7A). The guide slot 132 and the oval shaped openings 134 and 136 are longitudinally aligned along the center longitudinal axis of the bars 116, 118 and 120. The slot 132 is located in the portion of the guide bar assembly that extends distally from the coupling element 38.

Referring to FIG. 2, when the cutting blade assembly 22 is attached to the coupling element 38 of the oscillating tip saw 20, the guide bar openings 134 and 136 are aligned with the respective openings 102 in the cover 91 and the openings 104 in the coupling element 38. Each of the openings 134 and 136 accommodates a separate one of the fasteners 93 (FIG. 1) that secure the cover 91 to the coupling element 38. The fasteners 93 also hold the saw blade assembly 22 to the oscillating tip saw 20. As shown in FIG. 7A, the openings 134 and 136 in the cutting blade assembly 110 are oval to accommodate for manufacturing variations between the components of this invention.

In certain preferred embodiments, the cutting blade assembly 22 may be secured to the oscillating tip saw 20 without using the fasteners 93. In some of these embodiments, the blade assembly 22 may be fastened to the oscillating tip saw 20 by a detent or other capture arrangement that allows the proximal end of the cutting blade assembly 22 to be quickly fastened to the oscillating tip saw 20.

Referring to FIGS. 7A and 8, the guide bar assembly 110 preferably includes a pivot pin 140 formed of hardened metal, such as tungsten carbide. The pivot pin 140 is located immediately forward of the distal end of the inner bar 118 and extends between the respective bottom and top bars 116 and 120. More particularly, the bottom bar 116 and the top bar 120 are formed with respective holes 142 and 144, and the opposite ends of the pivot pin 140 are welded or otherwise secured in the holes 142 and 144.

After the bottom bar 116, the inner bar 118 and the top bar 120 are assembled together, the inner bar 118 and the support bars 130 hold the bottom bar 116 and the top bar 120 apart or in spaced relation to one another. The inner bar 118 and the support bars 130 also contribute to the overall rigidity of the guide bar assembly 110.

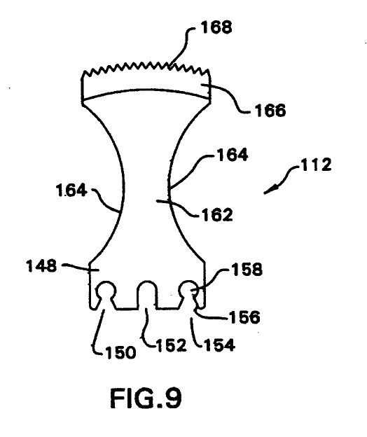

Referring to FIG. 9, in certain preferred embodiments of the present invention, the saw blade 112 includes a monolithic piece of metal, such as stainless steel. The saw blade 112 is shaped to have a generally rectangular base 148 that forms the proximal end of the blade 112. The base 148 is shaped to have three notches 150, 152 and 154 that extend forward from the proximal end of the blade 112. The central notch 152 is U-shaped and is located along the longitudinal central axis of the saw blade 112.

The two outer notches 150 and 154 are located on opposite sides of the central notch 152 and are equidistantly spaced from the longitudinal center axis of the saw blade 112. The outer notches 150 and 154 are preferably identically shaped. Each outer notch 150 and 154 has a tapered proximal section 156 having the greatest width adjacent the proximal end of the saw blade 112. Integral with and located forward of the proximal section 156, each outer notch 150 and 152 has a distal end 158 with a circular cross-sectional profile. The diameter of the distal end 158 of each outer notch is approximately equal to that of the widest width of the proximal sections 156 of the outer notches.

Extending forwardly from the base 148, the saw blade 112 has a main section 162 having two opposed sides 164 that have a concave curvature forming a narrow waist at a middle portion of the main section 162.

Distal from the main section 162, the saw blade 112 has an arcuately shaped tip 166 having teeth 168. It will be appreciated that the blade 112 need not be outwardly curved as shown, and instead may have other configurations. The distal tip 166 is preferably part of the cutting portion of the saw blade 112.

In certain preferred embodiments, the saw blade 112 has a side-to-side width of about 1.5 inches or less and preferably about 0.9 inches or less. The overall length of the saw blade 112, from the proximal end of the base 148 to the most distal tooth 168, is preferably about 3.0 inches or less and preferably about 1.5 inches or less.

Referring to FIGS. 7A and 8, the saw blade 112 is preferably disposed between the distal end sections of the bottom and top bars 116 and 120, respectively. The saw blade 112 is positioned between the bars 116 and 120 so that the pivot pin 140 is aligned with central notch 152. In operation, the saw blade 112 is pivotable about the pin 140.

Referring to FIG. 8, in certain preferred embodiments of the present invention, the bottom and top support bars 116, 120 are magnetic and the inner bar 118, the drive elements 114 and the cutting blade 112 are non-magnetic. As described above, the magnetic bottom and top support bars 116, 120 will attract the cutting blade assembly to a guide surface of a cutting guide so that the cutting blade assembly remains properly aligned during a bone cutting procedure. The magnetized bottom and top support bars 116, 120 will minimize the effects of vibration because the bars 116, 120 will be attracted to the guide surface of the cutting guide. The inner bar 118, the drive elements 114 and the cutting blade 112 are non-magnetized so that the elements are able to move freely during a bone cutting procedure. In other preferred embodiments, only the bottom support bar 116 is magnetic and the top support bar 120, the inner bar 118, the drive elements 114 and the cutting blade 112 are non-magnetic.

In yet another preferred embodiment of the present invention, the bottom and top support bars 116, 120 and the cutting blade 112 are magnetized and the inner bar 118 and the drive elements 114 are non-magnetized. The magnetized bottom and top support bars are attracted to the guide surfaces of cutting guides for ensuring that the cutting blade assembly remains properly aligned during a bone cutting procedure. The magnetic cutting blade can be tracked by a navigation tracker as it advances through bone. Thus, the exact location and alignment of the cutting blade can be observed during surgery.

Referring to FIG. 7B, in certain preferred embodiments of the present invention, the saw blade assembly 22 is constructed so that the blade tip 166 has a depth, i.e. a thickness, greater than that of the adjacent guide bar assembly 110. The saw blade 112 is shaped so that the opposed top and bottom surfaces of the blade tip 166 extend, respectively, above and below the adjacent top and bottom surfaces of the guide bar assembly 110. As a result of this design, when the saw 20 is actuated and the blade tip 166 pressed into the bone, the resultant kerf is slightly larger than the thickness of the guide bar assembly 110, which facilitates the movement of the guide bar assembly 110 through the kerf as the bone is cut. In practice, it is anticipated that the saw blade assembly 22 will be designed so that the blade tip 166 has a thickness that is approximately 0.010 inches greater than that of the guide bar assembly 110 from which the oscillating tip extends. The saw blade 112 is shaped so that the extra thickness of the saw tip 166 is symmetrically arranged relative to the top and bottom surfaces of the guide bar assembly 110. Thus, the blade tip 166 preferably extends approximately 0.005 inches beyond each of the top and bottom surfaces of the guide bar assembly.

Referring to FIG. 7B, in certain preferred embodiments, the guide bar assembly has a thickness of approximately 0.090 inches and the saw blade tip 166 has a thickness of approximately 0.100 inches. In other preferred embodiments, the thinnest saw blade assemblies 22 may have guide bar assemblies with a thickness of 0.040 inches and blade tips with thicknesses of 0.050 inches.

Referring to FIGS. 8 and 9, the oscillating tip saw includes drive rods 114 that connect the saw blade 112 to the opposed ends of the oscillating bar 86 (FIG. 2). The drive rods 114 are preferably formed of a metal such as 17-4 stainless steel. In certain preferred embodiments, the drive rods 114 are formed of a material having a slight degree of elasticity. The proximal end of each drive rod 114 has a ring 170 and the distal end of each drive rod 114 has a solid, circular shaped head 172.

Referring to FIGS. 2 and 8, when the oscillating tip saw is assembled together, each drive rod 114 is preferably located between the bottom and top outer bars 116 and 120 adjacent the sides of the inner bar 118. The proximal end ring 170 of each drive rod is fitted over a separate one of the pins 85 integral with the oscillating bar 86. The distal end head 172 is seated in the distal section 158 of the outer notches 150, 154 at the proximal end of the saw blade 112.

When disassembling the oscillating tip saw, the saw blade 112 is removed and replaced by first removing the cover 91 from the coupling element 38. The saw blade assembly 22 is then removed from the coupling element 38 so that the drive rod rings 170 are lifted off the pins 85. Once the guide bar assembly 110 is free of the coupling element 38, the saw blade 112 is pulled forward so as to expose the drive rod heads 172. Once the saw blade 112 has been pulled forward to expose the drive rod heads 172, the old blade 112 may be easily removed from the drive rods 114 and replaced with a new blade. Once the new blade has been attached to the drive rod heads 172, the blade and the drive rods may be urged proximally toward the coupling element 38 to a position where central notch 152 engages the pivot pin 140. The reassembled saw blade assembly is then reattached to the coupling element 38 by placing the proximal end rings 170 over the pins 85 and securing the cover 91 over the coupling element 38.

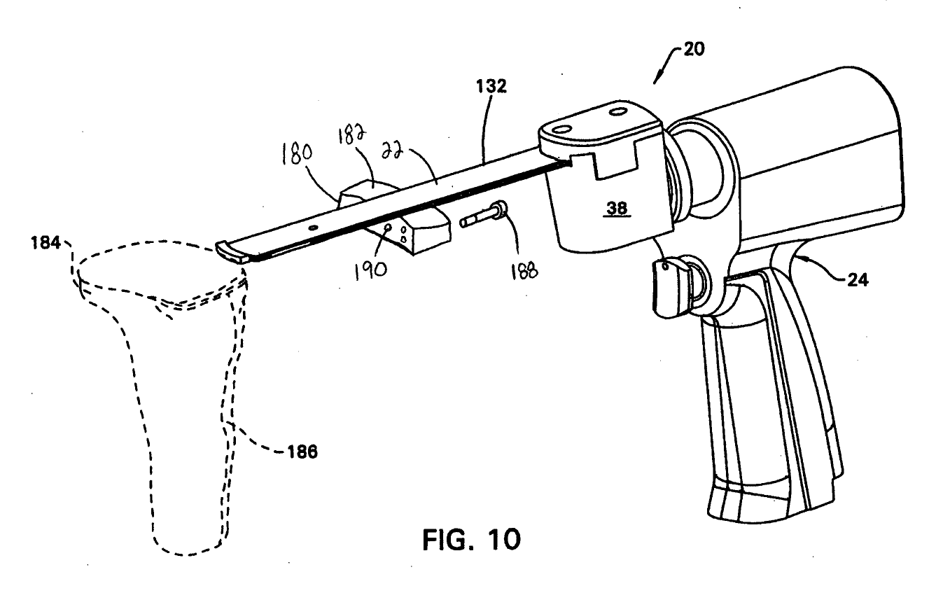

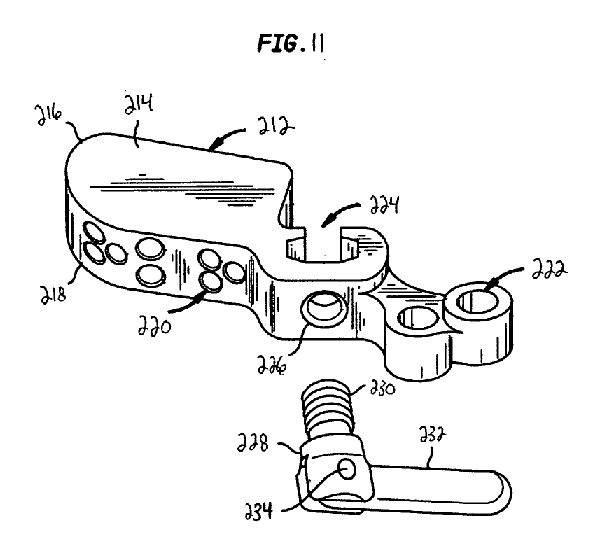

Referring to FIG. 10, in certain preferred embodiments of the present invention, a cutting guide 180 is used for guiding the oscillating tip saw 20 and the saw blade assembly 22 steady when making a desired cut in bone during a bone cutting procedure. The cutting guide 180 is preferably a metal block that is affixed to bone, preferably at a location adjacent a bone resection surface. The cutting guide 180 includes a guide surface 182 for guiding advancement of a saw blade or a cutting blade assembly as described above. In certain embodiments, the cutting guide 180 is temporarily fixed to the proximal end or head 184 of a tibia 186 (tibia represented diagrammatically). One or more pins 188 temporarily secure the cutting guide 180 to the tibia 186. The pins 188 are passed through bores 190 extending though the cutting guide and into the tibia 186.

The guide surface 182 of the cutting guide 180 aligns the cutting blade assembly 22 with designated cut lines through the bone 186. The magnetized elements of the cutting blade assembly insure that the cutting blade assembly is attracted to the guide surface, which in turn insures that the bone cuts are made along the designated cut lines. In certain preferred embodiments, both the cutting guide 180 and elements of the cutting blade assembly 22 are magnetized for generating the magnetic attraction between the guide surface 182 and the cutting blade assembly 22.

Referring to FIG. 11, in certain preferred embodiments of the present invention, a system for preparing bone for receiving an implant includes a cutting guide 212 having a top guide surface 214, an inner contoured surface 216 that is preferably shaped to fit against the bone and an outer contoured surface 218 that is adapted to fit easily within an incision. The cutting guide 212 shown in FIG. 11 is preferably used to perform a tibial resection at a proximal end of a tibia, however, it may be used for other orthopedic procedures as well. The cutting guide 212 desirably includes one or more holes 220 that may receive fasteners such as pins for securing the cutting guide to bone. One or more of the holes 220 may be adapted to secure a tool thereto, as will be described in more detail below. The cutting guide also preferably includes one or more openings 222 adapted to secure a navigation tracker for properly aligning the cutting guide to the proximal end of a tibia. In other preferred embodiments of the present invention, however, the cutting guide may be magnetized and the navigation tracker may be coupled with the cutting guide through magnetic attraction forces. Thus, the navigation tracker may be held to the cutting guide through magnetic forces and not mechanical connections.

The cutting guide 212 also preferably includes a C-shaped opening 224 engageable with an elongated element such as a rod. After the rod is coupled with the C-shaped opening 224, the cutting guide is designed to slide along the rod for adjusting the location of the cutting guide relative to bone, such as the proximal end of the tibia. The cutting guide 212 may also include a threaded opening 226 aligned with the C-shaped opening 224. A tightening screw 228 having threads 230 may be inserted into the threaded opening 226. The tightening screw 228 also preferably includes a lever 232 that may pivot about a pivot point 234 for enabling greater leverage to be applied to the tightening screw. The pivotable lever also preferably allows the screw 228 and the lever 232 to remain below the guide surface 214 of the cutting guide 212.

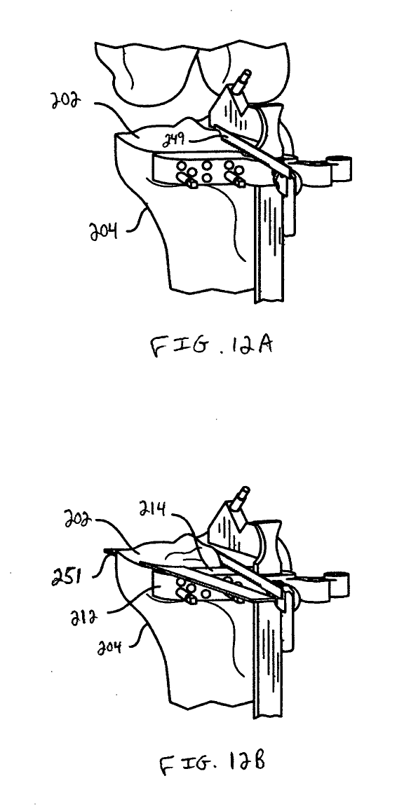

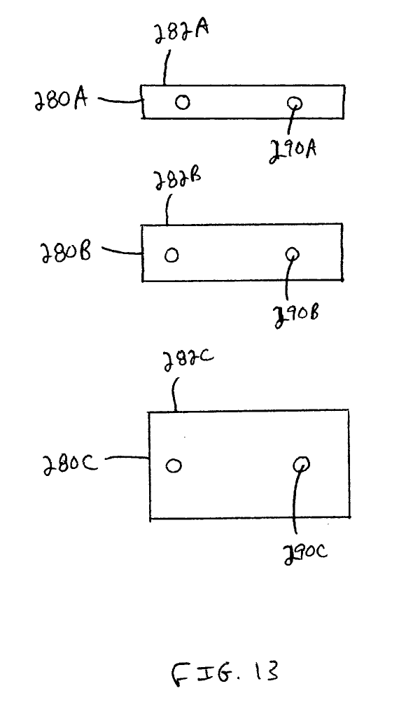

Referring to FIGS. 12A and 12B, a saw 249 or other cutting instrument may be used to make a saggital resection of the proximal end 202 of the tibia 204. Referring to FIG. 12B, a second magnetized saw 251 or cutting instrument may be used to cut the proximal end 202 of the tibia 204 in a plane defined by the top surface 214 of the tibial resection block 212. The magnetized saw 251 is attracted to the top guide surface 214 for guiding the saw along designated cut lines through the bone. The magnetic forces keep the saw blade 251 from becoming improperly aligned with the plane formed by the guide surface 214. In another preferred embodiment, both the saw 251 and the cutting guide 212 are magnetized for magnetically attracting the saw blade to the guide surface of the cutting guide.

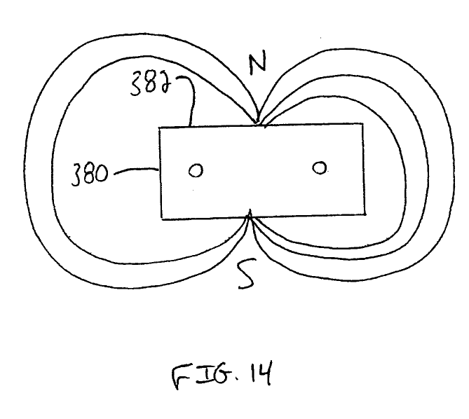

Referring to FIG. 13, in certain preferred embodiments of the present invention, a kit may include a plurality of cutting guides 280A-280C having different sizes. Each cutting guide has at least one guide surface 282A-282C for guiding a cutting instrument during a bone cutting procedure. The guide surfaces 282A-282C are preferably planar for properly aligning the cutting blade on the cutting instrument with the pre-designated cut lines in the bone or tissue. The cutting guides are attached to bone using the openings 290A-290C. The cutting guides are preferably made of metal for attracting the one or more magnetic elements on the cutting instrument so as to provide the benefits discussed above.

Referring to FIG. 14, in certain preferred embodiments of the present invention, a cutting guide 380 may be magnetic. In these particular embodiments, at least one of the elements on the cutting instrument and the cutting guide are magnetic for attracting at least one magnetic element to the guide surface 382 of the cutting guide. The magnetic attraction insures proper alignment and orientation of the cutting instrument with the pre-determined cut lines in bone during a bone cutting procedure.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for cutting bone comprising:
a cutting guide having a guide surface;
a cutting instrument including a cutting blade, wherein at least one element of said cutting instrument is magnetic for magnetically attracting said at least one element to said guide surface of said cutting guide.

2. The system as claimed in claim 1, wherein said cutting guide is made of metal.

3. The system as claimed in claim 2, wherein said magnetic cutting guide is magnetic.

4. The system as claimed in claim 1, wherein said at least one element comprises a magnetic saw blade.

5. The system as claimed in claim 1, wherein said cutting instrument comprises an oscillating tip saw and said at least one magnetic element is a guide bar assembly that supports an oscillating cutting blade.

6. The system as claimed in claim 5, wherein said oscillating cutting blade is non-magnetic.

7. The system as claimed in claim 1, wherein said cutting instrument comprises an oscillating tip saw including a cutting blade assembly having a magnetic portion and a non-magnetic portion.

8. The system as claimed in claim 7, wherein said magnetic portion of said cutting blade assembly is non-oscillating and said non-magnetic portion of said cutting blade assembly is adapted for selectively oscillating.

9. The system as claimed in claim 1, wherein said cutting instrument comprises:
a guide bar assembly having a proximal end and a distal end;
a cutting blade pivotally mounted to said guide bar assembly, wherein said cutting blade projects beyond the distal end of said guide bar assembly;
at least one drive element coupled with said cutting blade for selectively oscillating said cutting blade.

10. The system as claimed in claim 9, wherein said cutting blade has cutting teeth.

11. The system as claimed in claim 9, wherein said guide bar assembly is magnetic and said cutting blade is non-magnetic.

12. The system as claimed in claim 11, wherein said at least one drive element is non-magnetic.

13. The system as claimed in claim 9, wherein said guide bar assembly and said cutting blade are magnetic and said at least one drive element is non-magnetic.

14. The system as claimed in claim 13, wherein said cutting blade has cutting teeth.

15. The system as claimed in claim 9, wherein said guide bar assembly comprises:
a magnetic bottom bar;
a magnetic top bar spaced from said bottom bar;
a non-magnetic inner bar disposed between said bottom bar and said top bar, wherein said cutting blade projects from distal ends of said bottom bar and said top bar.

16. The system as claimed in claim 15, wherein said at least one drive element comprises a pair of non-magnetic drive rods that extend between said bottom bar and said top bar along opposite lateral sides of said inner bar.

17. The system as claimed in claim 1, wherein said cutting instrument comprises:
a housing;
a motor located in said housing and coupled with said cutting blade;
an actuator in communication with said motor for selectively activating said motor for driving oscillating movement of said cutting blade.

18. The system as claimed in claim 17, further comprising a drive shaft connected with said motor and coupled with said cutting blade, wherein said drive shaft drives oscillating movement of said cutting blade when said motor is activated.

19. A system for cutting bone comprising:
a cutting guide including a metal block having a guide surface provided thereon;
an oscillating tip saw having a cutting blade assembly extending from a distal end thereof, wherein at least one element of said cutting blade assembly is magnetic for generating a magnetic attraction to said guide surface so as to control the orientation of said cutting blade assembly during a bone cutting procedure.

20. The system as claimed in claim 19, wherein said cutting blade assembly comprises a magnetic guide bar assembly and a non-magnetic cutting blade pivotally connected with said magnetic guide bar assembly.

21. The system as claimed in claim 20, wherein said cutting blade assembly further comprises at least one non-magnetic drive element extending through said guide bar assembly for driving oscillating movement of said cutting blade.

22. A system for cutting bone comprising:
a plurality of cutting guides having different sizes, each said cutting guide including a metal block having a guide surface provided thereof;
an oscillating tip saw including a cutting blade assembly extending from a distal end thereof, said cutting blade assembly having an oscillating cutting blade provided at a distal end thereof, wherein at least one element of said cutting blade assembly is magnetic for generating a magnetic attraction between said at least one magnetic element and said guide surfaces of said cutting guides.

23. The system as claimed in claim 22, wherein said metal blocks are magnetic.

24. The system as claimed in claim 22, wherein said oscillating tip saw comprises:
a housing;
a motor disposed in said housing and coupled with cutting blade assembly for selectively driving said oscillating cutting blade; and
an actuator provided on said housing and coupled with said motor for activating said oscillating cutting blade.

25. A system for cutting bone comprising:
a cutting guide having a guide surface;
a cutting instrument including an oscillating portion and a non-oscillating portion;
said oscillating portion of said cutting assembly being non-magnetic;
said non-oscillating portion of said cutting assembly being magnetized for attracting said non-oscillating portion of said cutting assembly to said guide surface of said cutting guide for guiding said cutting assembly over said guide surface when cutting bone.

26. The system as claimed in claim 25, wherein said cutting instrument comprises an oscillating tip saw.

27. The system as claimed in claim 25, wherein said oscillating portion includes a cutting blade having cutting teeth.

28. The system as claimed in claim 25, wherein said oscillating portion of said cutting instrument is non-magnetic.
